(19) United States
(12) Patent Application Publication
(10) Pub. No.: US 2020/0337647 A1
(43) Pub. Date: Oct. 29, 2020

(54) METHOD AND ELECTRONIC DEVICE FOR PREDICTING SUDDEN DROP IN BLOOD PRESSURE

(71) Applicant: Wistron Corporation, New Taipei City (TW)

(72) Inventor: Yi-Shuan Chen, New Taipei City (TW)

(73) Assignee: Wistron Corporation, New Taipei City (TW)

(21) Appl. No.: 16/528,552

(22) Filed: Jul. 31, 2019

(30) Foreign Application Priority Data
Apr. 25, 2019 (TW) 108114581

(51) Int. Cl. A61B 5/00; A61B 5/021

(52) U.S. Cl. CPC A61B 5/7275; A61B 5/021; A61B 5/4836; A61B 5/746; A61B 5/7267

(57) ABSTRACT

A method for predicting sudden drop in blood pressure is provided. The method includes following steps: receiving first physiological information corresponding to a first user; receiving a first current blood pressure of the first user; obtaining a sudden drop probability according to a blood feature model, the first physiological information and the first current blood pressure; determining whether the sudden drop probability is not less than a trigger threshold; and determining that a sudden drop in blood pressure will occur in response to determining that the sudden drop probability is not less than the trigger threshold.

### CROSS-REFERENCE TO RELATED APPLICATION

This application claims the priority benefit of Taiwan application serial no. 108114581, filed on Apr. 25, 2019. The entirety of the above-mentioned patent application is hereby incorporated by reference herein and made a part of this specification.

### BACKGROUND

### Technical Field

The disclosure relates a prediction technology, and more particularly, to a method and an electronic device for predicting sudden drop in blood pressure.

### Description of Related Art

Hemodialysis is one of the common medical treatments. During hemodialysis, as blood will be drained to a dialysis machine and returned to the body, the patient may have a sudden drop in blood pressure due to dehydration and suffer physical discomforts. Worse yet, the sudden drop in blood pressure may have occurred for a while by the time the patient suffers physical discomforts. In addition, because each patient may have a different physical condition, when the patient is in a unstable condition, it is still necessary to rely on professional judgment and real-time monitoring of the medical personnel, which will then increase the medical cost. Accordingly, how to reduce discomforts for the patient and reduce the medical cost is one of the issues to be addressed by person skilled in the art.

### SUMMARY

The disclosure provides a method and an electronic device for predicting sudden drop in blood pressure that can predict a sudden drop in blood pressure in advance for the patient.

In an embodiment of the disclosure, the method for predicting sudden drop in blood pressure includes the following steps of: receiving first physiological information corresponding to a first user; receiving a first current blood pressure of the first user; obtaining a sudden drop probability according to a blood feature model, the first physiological information and the first current blood pressure; determining whether the sudden drop probability is not less than a trigger threshold; and determining that a sudden drop in blood pressure will occur in response to determining that the sudden drop probability is not less than the trigger threshold.

In an embodiment of the disclosure, the electronic device includes an input device, a storage device and a processor. The input device receives first physiological information and a first current blood pressure corresponding to a first user. The storage device stores a blood feature model. The processor is connected to the input device and the storage device, and obtains a sudden drop probability according to the blood feature model, the first physiological information and the first current blood pressure. The processor further determines whether the sudden drop probability is not less than a trigger threshold, and determines that a sudden drop in blood pressure will occur in response to determining that the sudden drop probability is not less than the trigger threshold.

Based one the above, in the electronic device for predicting sudden drop in blood pressure and the method for predicting sudden drop in blood pressure provided by the disclosure, whether the sudden drop in blood pressure will occur is predicted in advance according to the blood feature model. In this way, the medical personnel can arrange a treatment for the patient before the sudden drop in blood pressure occurs, so as to prevent the patient from the discomforts. In addition, the medical personnel can focus on those patients who really in need for attention so as to relief the burden on the medical personnel.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 is a schematic diagram illustrating an electronic device for predicting sudden drop in blood pressure according to an embodiment of the disclosure.

FIG. 2 is a schematic diagram illustrating a structure of an electronic device for predicting sudden drop in blood pressure according to an embodiment of the disclosure.

FIG. 3 is a flowchart illustrating a method for predicting sudden drop in blood pressure according to an embodiment of the disclosure.

FIG. 4 is a schematic diagram illustrating how a blood feature model is created according to an embodiment of the disclosure.

FIG. 5 illustrates flowchart illustrating a feedback operation according to an embodiment of the disclosure.

FIG. 6 is a schematic diagram illustrating an application of an electronic device in an embodiment of the disclosure.

### DESCRIPTION OF THE EMBODIMENTS

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

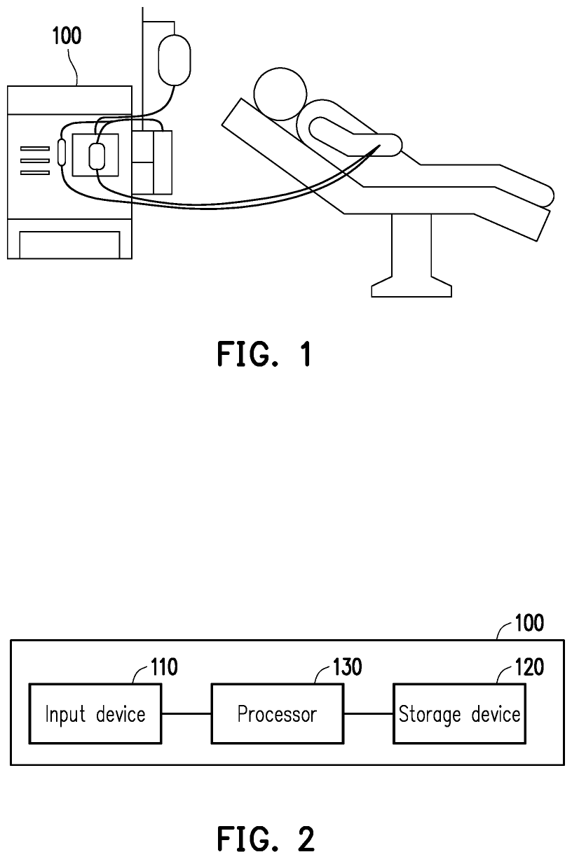

FIG. 1 is a schematic diagram illustrating an electronic device for predicting sudden drop in blood pressure according to an embodiment of the disclosure. With reference to FIG. 1, an electronic device 100 is applied in hemodialysis, and configured to predict whether the user will have a sudden drop in blood pressure. In an embodiment of the disclosure, the electronic device 100 may be a hemodialysis machine, a control instrument, or any electronic device capable of receiving physiological data of the user and performing computing functions. A type of the electronic device 100 is not particularly limited by the disclosure.

FIG. 2 is a schematic diagram illustrating a structure of an electronic device for predicting sudden drop in blood pressure according to an embodiment of the disclosure. With reference to FIG. 2, in an embodiment of the disclosure, the electronic device 100 at least includes an input device 110, a storage device 120 and a processor 130.

The input device 110 is configured to receive physiological information of the user. The physiological information of the user includes, for example, one or more of blood concentration, sodium concentration of blood, dry weight, hematocrit (HCT), insulin, urea nitrogen, creatinine, calcium, cholesterol, iron, but the disclosure is not limited thereto.

In an embodiment of the disclosure, the input device 110 may be a keyboard, a mouse, a touch panel and the like, which can allow a medical professional to input the physiological information of the user. In addition, the input device 110 may also be various measuring devices, such as a hemomanometer, a blood analyzer and the like, which can measure physiological parameters of the user and directly input the physiological parameters to the electronic device 100. Alternatively, the input device 110 may be various connection ports connected to various measuring devices, processing devices (e.g., personal computers) and the like, which can perform data transmission via the connection ports to obtain the physiological information of the user. In addition, the input device 110 may also a combination of the devices described above, or other devices capable of obtaining the physiological information of the user, which are not particularly limited by the disclosure.

The storage device 120 is configured to store various data and program codes required for operating the electronic device 100. In this embodiment, the storage device 120 may be a random access memory (RAM), a read-only memory (ROM), a flash memory, a hard Disk drive (HDD), a hard disk drive (HDD) as a solid state drive (SSD) or other similar devices in any stationary or movable form, or a combination of the above-mentioned devices, but the disclosure is not limited thereto.

The processor 130 is connected to the input device 110 and the storage device 120, and configured to execute various operations required by the electronic device 100. Further, the processor 130 may be, for example, a central processing unit (CPU) or other programmable devices for general purpose or special purpose such as a microprocessor and a digital signal processor (DSP), a programmable controller, an application specific integrated circuit (ASIC) or other similar elements or a combination of above-mentioned elements, but the disclosure is not limited thereto.

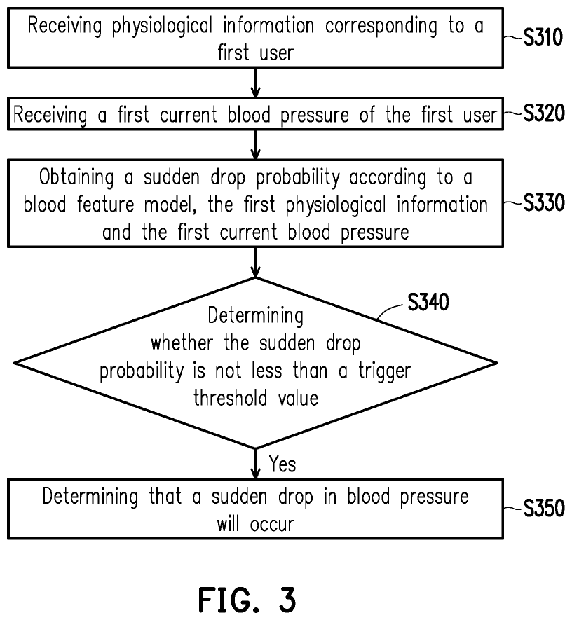

FIG. 3 is a flowchart illustrating a method for predicting sudden drop in blood pressure according to an embodiment of the disclosure. In this embodiment, the method for predicting sudden drop in blood pressure is at least adapted to the electronic device 100 in FIG. 1 and FIG. 2, but the disclosure is not limited thereto. Details regarding how the method for predicting sudden drop in blood pressure may be performed by the cooperation of the input device 110, the storage device 120 and the processor 130 of the electronic device 100 are described below with reference to FIG. 1 to FIG. 3.

In step S310, the processor 130 receives physiological information corresponding to a first user through the input device 110. Then, in step S320, the processor 130 receives a first current blood pressure of the first user through the input device 110. As described above, the input device 110 receives the physiological information corresponding to the user input by the medical professional or inputs the physiological information of the user obtained through measurement or through connection with other electronic devices to the electronic device 100, and details regarding the same are not repeated hereinafter.

In step S330, the processor 130 obtains a sudden drop probability according to a blood feature model, the first physiological information and the first current blood pressure. In detail, the blood feature model refers a rule for predicting a future blood pressure created in advance by ways of machine learning. Details regarding how the processor 130 creates and stores the blood feature model in the storage device 120 will be described later.

The sudden drop probability is used to indicate a probability of the sudden drop in blood pressure that will occur at a certain time point or a time interval in the future. In this embodiment, the sudden drop probability is a probability of the sudden drop in blood pressure that will occur in the next 30 minutes (e.g., 35%).

In step S340, the processor 130 determines whether the sudden drop probability is not less than a trigger threshold. In step S350, the processor 130 determines that a sudden drop in blood pressure will occur in response to determining that the sudden drop probability is not less than the trigger threshold. The trigger threshold is a criterion for determining whether the sudden drop in blood pressure will occur. If the sudden drop probability is not less than the trigger threshold, the processor 130 predicts that the patient will have the sudden drop in blood pressure. In other words, the trigger threshold being smaller means that even if the processor 130 determines that the probability of the sudden drop in blood pressure is very small, the processor 130 will still determine that the patient will have the sudden drop in blood pressure. For instance, if the trigger threshold is 35% and the probability of the sudden drop in blood pressure that the patient will have in the next 30 minutes is not less than 35%, the processor 130 determines that the sudden drop in blood pressure will occur. In an embodiment of the disclosure, the trigger threshold may be adjusted by the medical personnel and stored in the storage device 120, but the disclosure is not limited thereto.

It is worth noting that, when determining that the sudden drop in blood pressure will occur, the processor 130 can further send an alert notice. In this embodiment of the disclosure, a method for sending the alert notice may include, for example, playing an alert sound, displaying an alert message, sending the alert message to a nursing station or an electronic device owned by the medical personnel, and may be adjusted based on different designs of the electronic device 100. However, the disclosure is not limited in this regard.

In the method for predicting sudden drop in blood pressure performed through the input device 110, the storage device 120, and the processor 130 of the electronic device 100, the sudden drop in blood pressure may be predicted in advance before the patient has the sudden drop in blood pressure. In this way, the medical personnel can arrange a treatment for the patient before the sudden drop in blood pressure occurs, so as to prevent the patient from the discomforts. In addition, the medical personnel can focus on those patients who really in need for attention so as to relief the burden on the medical personnel.

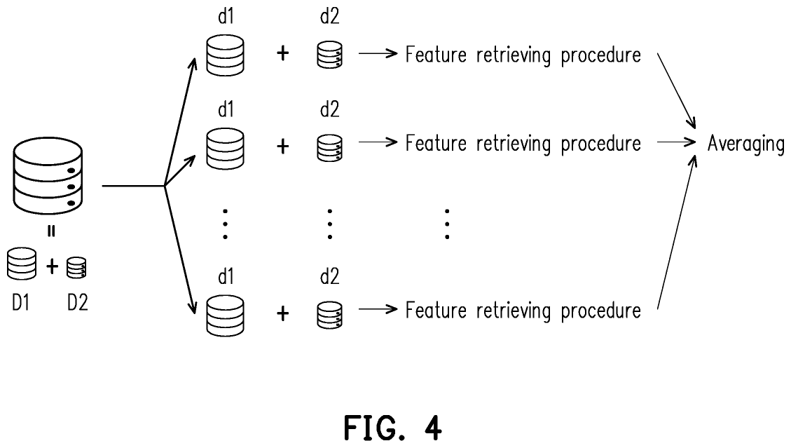

FIG. 4 is a schematic diagram illustrating how a blood feature model is created according to an embodiment of the disclosure. How the blood feature model is created by the processor 130 is described below with reference to FIG. 4.

Before starting to create the blood feature model, the processor 130 first obtains training data for creating the blood feature model. Specifically, each entry of the training data includes the physiological information from the patient and status information during hemodialysis, such as physiological data of the patient before hemodialysis, a blood pressure change during hemodialysis, physiological data after hemodialysis, blood pressure, etc. The processor 130 collects the training data based on “each hemodialysis performed”. That is to say, regardless of whether or not the training data of the same patient already exists, each hemodialysis performed for the same patient or different patients may be regarded as one entry of the training data, but the disclosure is not limited thereto.

In the clinical manifestations of hemodialysis, the number of patients not having the sudden drop in blood pressure was 17 times the number of patients having the sudden drop in blood pressure, which was imbalance data. Accordingly, if feature retrieving and averaging are performed on all the training data at the same time, a blood feature model that focuses more on the patient not having the sudden drop in blood pressure will be generated, misjudgments are likely to happen when that blood feature model is used to determine whether the sudden drop in blood pressure will occur. In order to level the difference in the imbalance data, first of all, according to whether the patients have the sudden drop in blood pressure during hemodialysis, the processor 130 groups the training data into a normal blood data group D1 and a sudden drop data group D2. Here, the normal blood data group D1 corresponds to the training data for the patients not having the sudden drop in blood pressure, and the sudden drop data group D2 corresponding to the training data for the patients having the sudden drop in blood pressure.

In detail, the processor 130 groups the training data according to a sudden drop rule. In this embodiment, the sudden drop rule includes, for example, three conditions listed in Table 1. In other words, the blood pressure change of the patient satisfying one of the three conditions below means that the patient has the sudden drop in blood pressure.

TABLE 1
the sudden drop rule (numerical unit: mmHg)

| | Previous blood pressure BP1 | Next blood pressure BP2 |
|---|---|---|
| Case 1 | BP1 ≤ 100 | BP2 ≤ BP1 × 90% |
| Case 2 | 100 < BP1 < 140 | BP2 ≤ BP1 × 50% + 40 |
| Case 3 | 140 ≤ BP1 | BP2 ≤ BP1 − 30 |

Previous blood pressure BP1 and Next blood pressure BP2 in Table 1 are blood pressures measured at intervals of 30 minutes. Alternatively, Previous blood pressure BP1 and Next blood pressure BP2 may be two closest data entries according to the measurement time, but the disclosure is not limited thereto. Case 1 indicates that the sudden drop in blood pressure occurs if Previous blood pressure BP1 is less than and equal to 100 mmHg and Next blood pressure BP2 is less than or equal to 90% of Previous blood pressure BP1. Case 2 indicates that the sudden drop in blood pressure occurs if Previous blood pressure BP1 is between 100 mmHg and 140 mmHg and Next blood pressure BP2 is less than or equal to 50% of Previous blood pressure BP1 plus 40 mmHg. Case 3 indicates that the sudden drop in blood pressure occurs if Previous blood pressure BP1 is greater than and equal to 140 mmHg and Next blood pressure BP2 is less than or equal to Previous blood pressure BP1 minus 30 mmHg. In other words, because each person's physiological information is different, a threshold for determining the sudden drop in blood pressure will also be different for different patients. It should be noted that in other embodiments of the disclosure, the cases of the sudden drop in blood pressure may also be adjusted according to actual design requirements and professional medical knowledge, and the disclosure is not limited thereto. In this embodiment, the sudden drop rule is stored in the storage device 120 in advance.

Next, the processor 130 selects a first quantity of data and a second mount of data from the normal blood data group D1 and the sudden drop data group D2 respectively as a first data set d1 and a second data set d2, and trains the first data set d1 and the second data set d2 to obtain sudden drop features.

Since the sudden drop data group D2 include the data that truly require attentions, in this embodiment, the first quantity will be less than or equal to the second quantity to enhance an intensity of features from the second data set d2. In other embodiments, the first quantity may also be slightly greater than the second quantity (e.g., the first quantity is 55 and the second quantity is 50). However, when a ratio of the first quantity to the second quantity approaches 1, the intensity of features obtained from the second data set d2 is higher so the obtained sudden drop features can better represent the second data set d2.

Specifically, if the normal blood data group D1 includes 950 data entries in total and the sudden drop data group D2 includes 50 data entries in total, the processor 130 will select all the data in the sudden drop data group D2 as the second data set d2, i.e., the second quantity is 50. Also, the processor 130 sets the first quantity to be the same as the second quantity (i.e., the first quantity is also 50), and selects 50 data entries from the normal blood data group D1 as the first data set d1. Alternatively, the processor 130 may set the first quantity and the second quantity to constant values (e.g., the first quantity and the second quantity are both 50, or are respectively 60 and 50, 40 and 50, 50 and 30, 20 and 50, etc.). Alternatively, the processor 130 may also set the ratio of the first quantity to the second quantity to, for example, 1:1, 1.2:1, 1:2, 1:3, etc., and set the second quantity to a constant value (e.g., a data quantity of the sudden drop data group D2). Nonetheless, how the first quantity and the second quantity are set is not particularly limited by the disclosure. In addition, the processor 130 may, for example, randomly select the first quantity of data and the second quantity of data from the normal blood data group D1 and the sudden drop data group D2 according to a random sampling, or may divide the normal blood data group D1 and the sudden drop data group D2 into multiple groups by ways of random distribution and the like and select one of the groups. However, the disclosure is not limited in this regard.

The processor 130 performs a feature retrieving procedure on the first data set d1 and the second data set d2. In detail, the processor 130 performs an operation on the second data set d2 and one of the first data set d1 according to an adaptive boosting (Adaboost) algorithm for obtaining the sudden drop features, but the disclosure is not limited thereto.

In an embodiment of the disclosure, the processor 130 will obtain different first data sets d1 for performing the operation with the second data set d2 multiple times, and one sudden drop feature will be obtained from each operation. Lastly, the processor 130 calculates an average value of all the sudden drop features to generate the blood feature model.

Further, a result of the blood feature model is evaluated by adopting a sensitivity, a false omission rate (FOR) and a false positive rate (FPR) in this embodiment. Because the sensitivity refers to a proportion of cases predicted according to the blood feature model in which the sudden drop in blood pressure occurs among all cases in which the sudden drop in blood pressure actually occurs, the sensitivity is preferably to be as high as possible. Because the false omission rate refers to a proportion of cases in which the sudden drop actually occurs among all predicted cases in which the sudden drop actually does not occur, the false omission rate is preferably to be as low as possible. Because the false positive rate refers to a proportion of cases in which the sudden drop actually occurs among all cases in which the sudden drop does not actually occur, the false positive rate is preferably to be as low as possible. Among these indicators for evaluation, the sensitivity is the main indicator to be considered of. In a practical experiment, in the blood feature model created according to the embodiment of FIG. 4, when the trigger threshold is set to 0.35, the sensitivity can reach 90.08% while the false omission rate is 1.07% and the false positive rate is 54.83%.

However, it should be noted that, in other embodiments of the disclosure, how the blood feature model is created is not limited to the method described above. In other embodiments of the disclosure, the processor 130 may also increase the data quantity of the sudden drop data group D2 by using an interpolation to make the data quantity of the sudden drop data group D2 closer to a data quantity of the normal blood data group D1. However, the disclosure is not limited in this regard.

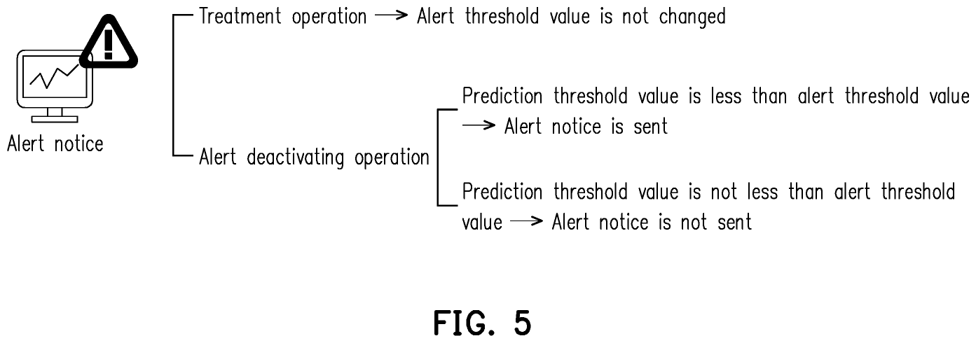

It is worth noting that, in an embodiment of the disclosure, the medical personnel may further perform a feedback operation for the alert notice provided by the electronic device 100. FIG. 5 illustrates flowchart illustrating a feedback operation according to an embodiment of the disclosure.

When receiving the first physiological information and an initial blood pressure, the processor 130 further calculates an alert threshold according to the blood feature model, the first physiological information, the initial blood pressure and the sudden drop probability. In detail, the alert threshold is a threshold used for determining that the sudden drop occurs. If the blood pressure of the user drops to the alert threshold, it means that the sudden drop in blood pressure occurs. In other words, the sudden drop probability may be regarded as a probability of a transition from the first current blood pressure to the alert threshold. For instance, if the first current blood pressure is 120 mmHg and the processor 130 determines that the sudden drop in blood pressure will occur when the blood pressure of the patient drops to 102 mmHg through calculation with the blood feature model, the alert threshold is set to 102 mmHg.

When evaluation of the blood pressure of the patient indicates that a probability of the blood pressure dropping to the alert threshold in the next 30 minutes is not less than the trigger threshold, the processor 130 will send the alert notice. At this point, the medical personnel can further determine whether the patient needs a further medical treatment according to his or her professional knowledge. If the alert notice is determined as true, it means that the patient is likely to have the sudden drop in blood pressure in the future based on the current blood pressure, i.e., the patient needs the medical treatment. In this case, the medical personnel may further press a “treatment” button.

When the processor 130 receives such a “treatment” operation, because the alert threshold is reliable, the processor 130 remains original settings for the alert threshold.

However, if the alert notice is determined as false, it means that the sudden drop in blood pressure may not occur based on the current physiological parameters of the patient. In this case, the medical personnel may then press an “alert deactivating” button. When the processor 130 receives an alert deactivating operation, it means that the alert threshold may be incorrect, or an estimation for the blood pressure after 30 minutes may be incorrect. Accordingly, the processor 130 re-adjusts the blood feature model according to the first physiological information, the initial blood pressure and the first current blood pressure, and generates a prediction threshold according to the first physiological information, the initial blood pressure, the first current blood pressure and the adjusted blood feature model. The prediction threshold indicates a blood pressure value that may be reached in the next 30 minutes based on the current blood pressure. In other words, the prediction threshold represents an estimation in the next 30 minutes from the current time point, and the alert threshold is a criterion for determining that the sudden drop in blood pressure will occur according to the blood feature model. If the prediction threshold is not less than the alert threshold, it means that, with the feedback from the medical personnel and the blood feature model adjusted according to the first current blood pressure, the future blood pressure estimated by the processor 130 is not less than the alert threshold. Therefore, the processor 130 stops sending the alert. However, if the prediction threshold is less than the alert threshold, it means that, with the feedback from the medical personnel, the future blood pressure estimated by the processor 130 is less than the alert threshold so that the sudden drop in blood pressure may still occur. Therefore, the processor 130 continues to send the alert notice.

With the feedback from the medical personnel in real time, the electronic device 100 can adjust the blood feature model at anytime to optimize performance of the blood feature model.

It is worth noting that in one experiment, the method for predicting sudden drop in blood pressure of the present embodiment and the existing regression model currently used are simulated to evaluate results of the two methods. According to the above description, the sensitivity is the most important indicator for the medical personnel when evaluating whether the patient will have the sudden drop in blood pressure. Therefore, based on the sensitivity of the regression model being 22.67%, the experiment is designed to evaluate performance of other variables in the case where the sensitivity used in this embodiment is also set to 22.67%. In other words, in this experiment, the trigger threshold in the method for predicting sudden drop in blood pressure will make overall performance fall at the sensitivity of 22.67%. In this way, with both sensitivities of the method for predicting sudden drop in blood pressure of the present embodiment and the existing regression model set to 22.67%, performance after adjustment by the medical personnel may then evaluated.

In the regression model, the sensitivity is originally 22.67%, and after the adjustment is made according to the feedback of the medical personnel, the sensitivity drops to 19.36% while the false omission rate is 5.33% and the false positive rate is 13.02%. Further, the number of the alert notices sent is 2268.

In the method for predicting sudden drop in blood pressure of the present embodiment, the originally sensitivity becomes 23.38% while the false omission rate is 4.65%, the false positive rate is 4.48% and the number of the alert notices sent is 943. In other words, compared to the existing regression model, the method for predicting sudden drop in blood pressure of the present embodiment may be used to not only improve the sensitivity but also lower the false omission rate and the false positive rate at the same time. Moreover, the number of the alert notices sent is reduced to 41% of the number of the alert notices sent when the regression model is adopted, so as to effectively relief the burden on the medical personnel.

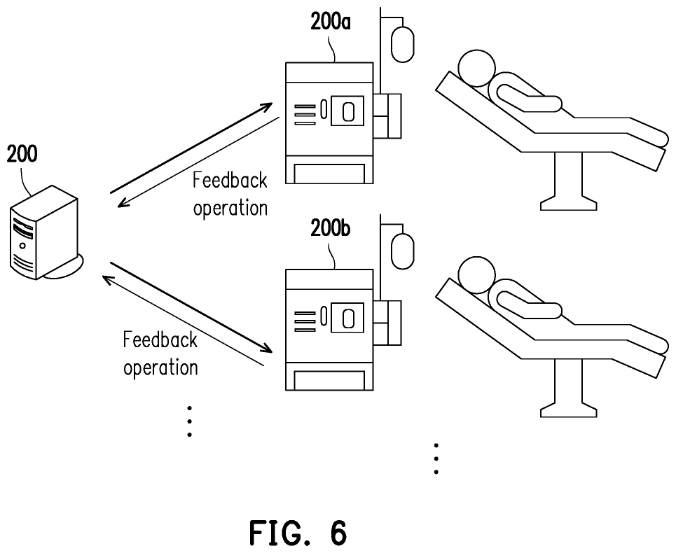

FIG. 6 is a schematic diagram illustrating an application of an electronic device in an embodiment of the disclosure. With reference to FIG. 6, in this embodiment, the method for predicting sudden drop in blood pressure is adapted to a cloud electronic device 200, a first electronic device 200a and a second electronic device 200b. Further, each of the cloud electronic device 200, the first electronic device 200a and the second electronic device 200b may be implemented by using the electronic device 100 in FIG. 1 and FIG. 2, but the disclosure is not limited thereto. After the first electronic device 200a adopts the method for predicting sudden drop in blood pressure to predict the sudden drop in blood pressure for the first user and receives the feedback operation of the medical personnel (e.g., receiving the treatment operation, the alert deactivating operation or other input information, which are not particularly limited by the disclosure), the first electronic device 200a transmits the adjusted blood feature model to the second electronic device 200b via the cloud electronic device 200. In this way, the second electronic device 200b can adjust a blood feature model by the second electronic device 200b according to the adjusted blood feature model.

It is worth mentioning that, in an embodiment of the disclosure, the cloud electronic device 200 only acts as a medium that transmits the adjusted blood feature model to first electronic device 200a or the second electronic device 200b. After receiving the adjusted blood feature model from the cloud electronic device 200, the first electronic device 200a and the second electronic device 200b will respective perform operations for optimizing their own stored blood feature models.

Nevertheless, in another embodiment of the disclosure, the cloud electronic device 200 also acts as a combiner that combines the adjusted blood feature models from both the first electronic device 200a and the second electronic device 200b to obtain an optimized blood feature model, and transmits the optimized blood feature model to the first electronic device 200a and the second electronic device 200b. The disclosure is not limited in this regard.

In summary, in the electronic device for predicting sudden drop in blood pressure and the method for predicting sudden drop in blood pressure provided by the disclosure, whether the sudden drop in blood pressure will occur is predicted in advance according to the blood feature model. In this way, the medical personnel can arrange a treatment for the patient before the sudden drop in blood pressure occurs, so as to prevent the patient from the discomforts. In addition, the medical personnel can focus on those patients who really in need for attention so as to relief the burden on the medical personnel. Moreover, the electronic device for predicting sudden drop in blood pressure and the method for predicting sudden drop in blood pressure can adjust the blood feature model and the evaluation of the sudden drop in blood pressure in real time according to the feedback from the medical personnel, so as to be immediately adapted to physical conditions of the patient and improve performance for predicting sudden drop in blood pressure. The adjusted blood feature model may be further used in other electronic devices for mutual learning to improve the overall performance of the blood feature model.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A method for predicting sudden drop in blood pressure, comprising:
receiving first physiological information corresponding to a first user;
receiving a first current blood pressure of the first user;
obtaining a sudden drop probability according to a blood feature model, the first physiological information and the first current blood pressure;
determining whether the sudden drop probability is not less than a trigger threshold; and
determining that a sudden drop in blood pressure is going to occur in response to determining that the sudden drop probability is not less than the trigger threshold.

2. The method for predicting sudden drop in blood pressure of claim 1, further comprising:
obtaining a plurality of sudden drop features according to a feature retrieving procedure, wherein each of the sudden drop features is retrieved from a second data set and one of a plurality of first data sets; and
obtaining the blood feature model by averaging the sudden drop features.

3. The method for predicting sudden drop in blood pressure of claim 2, further comprising:
selecting a first quantity of data from a normal blood data group as the first data sets; and
selecting a second quantity of data from a sudden drop data group as the second data set, wherein the first quantity is identical to the second quantity.

4. The method for predicting sudden drop in blood pressure of claim 2, wherein the feature retrieving procedure is to perform an operation on said second data set and one of the first data sets according to an adaptive boosting (Adaboost) algorithm for obtaining the sudden drop features.

5. The method for predicting sudden drop in blood pressure of claim 3, further comprising:
receiving a plurality of training data; and
determining whether the training data is classified to the normal blood data group or the sudden drop data group according to a sudden drop rule.

6. The method for predicting sudden drop in blood pressure of claim 1, further comprising: sending an alert notice in response to determining that the sudden drop in blood pressure is going to occur.

7. The method for predicting sudden drop in blood pressure of claim 6, further comprising:
receiving an initial blood pressure of the first user; and
obtaining an alert threshold according to the blood feature model, the first physiological information, the initial blood pressure and the trigger threshold.

8. The method for predicting sudden drop in blood pressure of claim 7, further comprising:
receiving a treatment operation; and
remaining the alert threshold according to the treatment operation.

9. The method for predicting sudden drop in blood pressure of claim 7, further comprising:
receiving an alert deactivating operation, and adjusting the blood feature model according to the first physiological information, the initial blood pressure and the first current blood pressure;
generating a prediction threshold according to the blood feature model, the first physiological information, the first current blood pressure, the trigger threshold and the adjusted blood feature model;
determining whether the prediction threshold is less than the alert threshold;
sending the alert notice in response to the prediction threshold being less than the alert threshold; and
not sending the alert notice in response to the prediction threshold not being less than the alert threshold.

10. The method for predicting sudden drop in blood pressure of claim 9, wherein the method for predicting sudden drop in blood pressure is adapted to a first electronic device and a second electronic device, and the method further comprises:
Transmitting, by the first electronic device, the adjusted blood feature model to the second electronic device; and
adjusting, by the second electronic device, a second blood feature model stored in the second electronic device according to the adjusted blood feature model.

11. An electronic device for predicting sudden drop in blood pressure, comprising:
an input device, receiving first physiological information and a first current blood pressure corresponding to a first user;
a storage device, storing a blood feature model; and
a processor, connected to the input device and the storage device, and obtaining a sudden drop probability according to the blood feature model, the first physiological information and the first current blood pressure, wherein
the processor determines whether the sudden drop probability is not less than a trigger threshold, and
wherein the processor determines that a sudden drop in blood pressure is going to occur in response to determining that the sudden drop probability is not less than the trigger threshold.

12. The electronic device of claim 11, wherein
the processor obtains a plurality of sudden drop features according to a feature retrieving procedure,
wherein each of the sudden drop features is retrieved from a second data set and one of a plurality of data sets, and
wherein the processor averages the sudden drop features to obtain the blood feature model.

13. The electronic device of claim 12, wherein
the processor selects a first quantity of data from a normal blood data group as of the first data set, and selects a second quantity of data from a sudden drop data group as the second data set, and
wherein the first quantity is identical to the second quantity.

14. The electronic device of claim 12, wherein the feature retrieving procedure is to perform an operation on said second data set and one of the first data sets according to an adaptive boosting (Adaboost) algorithm for obtaining the sudden drop features by the processor.

15. The electronic device of claim 12, wherein
the input device receives a plurality of training data, and
wherein the processor determines whether the training data is classified to the normal blood data group or the sudden drop data group according to a sudden drop rule.

16. The electronic device of claim 11, wherein the processor sends an alert notice in response to determining that the sudden drop in blood pressure is going to occur.

17. The electronic device of claim 16, wherein the input device receives an initial blood pressure of the first user; and
wherein the processor obtains an alert threshold according to the blood feature model, the first physiological information, the initial blood pressure and the trigger threshold.

18. The electronic device of claim 17, wherein the input device receives a treatment operation; and
wherein the processor remains the alert threshold according to the treatment operation.

19. The electronic device of claim 17, wherein
the input device receives an alert deactivating operation,
the processor adjusts the blood feature model according to the first physiological information, the initial blood pressure and the first current blood pressure,
the processor generates a prediction threshold according to the blood feature model, the first physiological information, the first current blood pressure, the trigger threshold and the adjusted blood feature model,
the processor determines whether the prediction threshold is less than the alert threshold,
wherein the processor sends the alert notice in response to the prediction threshold being less than the alert threshold, and does not send the alert notice in response to the prediction threshold not being less than the alert threshold.

20. The electronic device of claim 19, wherein the electronic device is communicated with a second electronic device,
wherein the processor transmits the adjusted blood feature model to the second electronic device such that the second electronic device adjusts a second blood feature model stored in the second electronic device according to the adjusted blood feature model.
